(19) United States
(12) Patent Application Publication
(10) Pub. No.: US 2026/0125343 A1
(43) Pub. Date: May 7, 2026

(54) METHODS FOR PREPARING HIGH-PURITY VORINOSTAT AND PRECURSORS THEREOF

(71) Applicant: UNRAVEL BIOSCIENCES, INC., MEDFORD, MA (US)

(72) Inventors: SHAILESHKUMAR RAMANLAL DESAI, WILMINGTON, NC (US); AUTUMN R. BEAUCHAMP, WILMINGTON, NC (US)

(21) Appl. No.: 19/382,407

(22) Filed: Nov. 7, 2025

Related U.S. Application Data
(60) Provisional application No. 63/717,313, filed on Nov. 7, 2024.

(51) Int. Cl. C07C 259/06; A61K 31/167

(52) U.S. Cl. CPC C07C 259/06; A61K 31/167

(57) ABSTRACT

The subject invention provides methods for synthesizing suberoylanilide hydroxamic acid (SAHA) and precursors thereof. These methods involve novel synthetic scheme for SAHA which eliminate purification of intermediates, and produce SAHA product in high purity and high yield, making them suitable for scale-up and GMP manufacturing of SAHA.

### CROSS-REFERENCE TO RELATED APPLICATION

This application claims the benefit of U.S. Provisional Application Ser. No. 63/717,313, filed Nov. 7, 2024, the disclosure of which is hereby incorporated by reference in its entirety, including all figures, tables and amino acid or nucleic acid sequences.

### BACKGROUND OF INVENTION

Vorinostat, also known as suberoylanilide hydroxamic acid (SAHA), is a small molecule inhibitor of histone deacetylases (HDACs). These enzymes play a crucial role in regulating chromatin structure and gene expression, and deregulation of HDAC activity has been implicated in various diseases including cancer. By selectively inhibiting HDACs, Vorinostat have multiple effects in vivo and in vitro, which includes arresting growth, affecting cell differentiation, and promoting the accumulation of acetylated histones, thereby altering gene transcription and inducing apoptosis in malignant cells.

The inhibition of HDAC by SAHA is thought to occur through direct interaction with the catalytic site of the enzyme as demonstrated by X-ray crystallography studies, which show that Vorinostat binds to the zinc atom of the catalytic site of the HDAC enzyme with the phenyl ring of Vorinostat projecting out of the catalytic domain onto the surface of the HDAC enzyme.

Vorinostat has been approved by the U.S. Food and Drug Administration (FDA) for the treatment of cutaneous T-cell lymphoma (CTCL) in patients who have received at least one prior systemic therapy, and preclinical studies also suggest that it may have therapeutic potential in other types of cancer, including breast, prostate, lung, and colon cancer.

While the synthesis of Vorinostat has been published over the last thirty years, the overall yields are generally low with varying purity levels.

U.S. Pat. No. 8,450,372B2, U.S. Pat. No. 7,851,509B2, and Stowel et al. (“The Synthesis of N-hydroxy-N′-Phenyoctanediamide and its Inhibitory Effect on proliferation of AXC Rat Prostate Cancer Cells,” J. Med. Chem. 1995, 38, 1411-1413) described processes for synthesizing SAHA with suberic acid or suberanilic acid as the starting material. However, SAHA was only produced in low yields of about 24%-36%.

The processes described in U.S. Pat. No. 5,369,108, Gediya et al. (“A New Simple and High-Yield Synthesis of Suberoylanilide Hydroxamic Acid and Its Inhibitory Effect Alone or in Combination with Retinoids on Proliferation of Human Prostate Cancer Cells,” J. Med. Chem. 2005, 48, 5047-5051), Truong et al. (“Modified Suberoylanilide Hydroxamic Acid Reduced Drug-Associated Immune Cell Death and Organ Damage under Lipopolysaccharide Inflammatory Challenge,” ACS Pharmacol. Transl. Sci. 2022, 5, 1128-1141), and Riva et al. (“Efficient Continuous Flow Synthesis of Hydroxamic Acids and Suberoylanilide Hydroxamic Acid Preparation,” J. Org. Chem. 2009, 74, 3540-3543), although increase the yield of SAHA to some extents, require chromatographic purification of the intermediates and/or the SAHA product, which are time-consuming, laborious, and environmentally unfriendly due to the use of large volumes of organic solvents.

Thus, it is desirable to provide a new route for preparing high-purity Vorinostat with improved yields in simplified procedures, which is suitable for scale-up to produce larger quantities and Good Manufacturing Practice (GMP) manufacturing.

### BRIEF SUMMARY OF INVENTION

The subject invention provides methods for synthesizing high-purity suberoylanilide hydroxamic acid (SAHA or Vorinostat). Advantageously, the synthesis procedure described in the present invention uses commercially available, low-cost materials (e.g., suberic acid monomethyl ester as starting material). The synthesis of Vorinostat includes easy work ups and eliminates the need to purify intermediates. A critical step in the synthesis is the generation of hydroxylamine using sodium methoxide, which avoids the use of water that typically generates poor yields. The isolated Vorinostat using this synthesis approach is produced in very high yields (80%-90%), and is a high purity material (>99%) that is suitable for use as a drug substance.

In one embodiment, the present invention provides a method for preparing SAHA from suberic acid monomethyl ester, the method comprising steps of:
(a) halogenation of suberic acid monomethyl ester to obtain methyl-8-halogeno-8-oxooctanoate;
(b) amidation of methyl-8-halogeno-8-oxooctanoate to obtain suberanilic acid methyl ester; and
(c) hydroxylamination of suberanilic acid methyl ester to obtain SAHA.

In some embodiments, the method further comprises a step of: (d) non-chromatographic purification of SAHA.

In some embodiments, step (a) of the method is carried out by reacting suberic acid monomethyl ester with one or more halogenation reagents in a molar ratio of suberic acid monomethyl ester: halogenation reagent of about 1:(1-2), and/or step (a) of the method is carried out at a temperature of about 20-80° C.

In some embodiments, step (b) of the method is carried out by reacting methyl-8-halogeno-8-oxooctanoate with aniline in the presence of one or more alkaline catalysts in a molar ratio of theoretical methyl-8-halogeno-8-oxooctanoate: aniline: alkaline catalyst of about 1:(1-2):(1-2), and/or step (b) of the method is carried out by firstly mixing the one or more alkaline catalysts with aniline, followed by adding the mixture of the one or more alkaline catalysts and aniline into methyl-8-halogeno-8-oxooctanoate.

In some embodiments, step (c) of the method is carried out by reacting suberanilic acid methyl ester with hydroxylamine in the presence of one or more alkaline catalysts in a molar ratio of theoretical suberanilic acid methyl ester: hydroxylamine: alkaline catalyst of about 1:(1-10):(1-10), and/or step (c) of the method is carried out by firstly mixing the one or more alkaline catalysts with hydroxylamine, followed by adding suberanilic acid methyl ester into the mixture of the one or more alkaline catalysts and hydroxylamine.

In some embodiments, step (a) and step (b) of the method comprise no chromatographic purification or non-chromatographic purification of intermediate obtained in each step.

In one embodiment, the present invention also provides a method for preparing SAHA from methyl-8-halogeno-8-oxooctanoate, the method comprising steps of:
(a) amidation of methyl-8-halogeno-8-oxooctanoate to obtain suberanilic acid methyl ester; and
(b) hydroxylamination of suberanilic acid methyl ester to obtain SAHA.

In some embodiments, the method further comprises a step of: (c) non-chromatographic purification of SAHA.

In some embodiments, methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

In some embodiments, step (a) of the method is carried out by reacting methyl-8-halogeno-8-oxooctanoate with aniline in the presence of one or more alkaline catalysts in a molar ratio of methyl-8-halogeno-8-oxooctanoate: aniline: alkaline catalyst of about 1:(1-2):(1-2).

In some embodiments, step (b) of the method is carried out by reacting suberanilic acid methyl ester with hydroxylamine in the presence of one or more alkaline catalysts in a molar ratio of theoretical suberanilic acid methyl ester: hydroxylamine: alkaline catalyst of about 1:(1-10):(1-10).

In some embodiments, the method comprises no chromatographic purification or non-chromatographic purification of intermediate obtained in the steps.

In one embodiment, the present invention further provides a method for preparing suberanilic acid methyl ester from suberic acid monomethyl ester, the method comprising steps of:
(a) halogenation of suberic acid monomethyl ester, to obtain methyl-8-halogeno-8-oxooctanoate; and
(b) amidation of methyl-8-halogeno-8-oxooctanoate, to obtain suberanilic acid methyl ester.

In some embodiments, step (a) of the method is carried out by reacting suberic acid monomethyl ester with one or more halogenation reagents in a molar ratio of suberic acid monomethyl ester: halogenation reagent of about 1:(1-2), and/or step (a) of the method is carried out at a temperature of about 20-80° C.

In some embodiments, step (b) of the method is carried out by reacting methyl-8-halogeno-8-oxooctanoate with aniline in the presence of one or more alkaline catalysts in a molar ratio of theoretical methyl-8-halogeno-8-oxooctanoate: aniline: alkaline catalyst of about 1:(1-2):(1-2), and/or step (b) of the method is carried out by firstly mixing the one or more alkaline catalysts with aniline, followed by adding the mixture of the one or more alkaline catalysts and aniline into methyl-8-halogeno-8-oxooctanoate.

### BRIEF DESCRIPTION OF DRAWINGS

FIG. 1 shows Mass Spectrum for Vorinostat Lot QCL-YS-041724.

FIG. 2 shows 1H-NMR Spectrum for Vorinostat Lot QCL-YS-041724.

FIG. 3 shows FTIR Spectrum for Vorinostat Lot QCL-YS-041724.

FIG. 4 shows FTIR Spectrum for Vorinostat Ambeed Reference Material Lot A234507-AA8.

FIGS. 5A-5B show On-scale and Off-scale Chromatogram of Vorinostat Lot QCL-YS-041724.

### DETAILED DESCRIPTION OF INVENTION

The present invention relates to novel processes for synthesizing the anti-cancer agent suberoylanilide hydroxamic acid (SAHA or Vorinostat) or a pharmaceutically acceptable salt thereof with optimized synthetic scheme and simplified processing procedures. SAHA can be prepared, via the synthesis process of the subject invention, in both higher purity and higher yield comparing to any existing preparation methods in the art, making the processes especially suitable for larger quantity scale-up manufacturing of SAHA and analytical qualification of GMP product.

As used herein and unless otherwise specified, the abbreviation “SAHA” or the term “Vorinostat” refers to suberoylanilide hydroxamic acid, which has a formula of C₁₄H₂₀N₂O₃, a molecular weight of 264.32 g/mol, and a structure of:

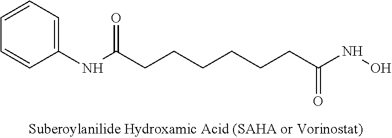 [SMILES: O=C(CCCCCCC(=O)Nc1ccccc1)NO]

Aiming to improve the production of SAHA, the present invention provides a scheme for synthesizing SAHA as illustrated below, where suberic acid monomethyl ester is used as the starting material, which is sequentially halogenated, amidated, and hydroxylaminated to produce the final product SAHA. The following synthetic scheme is determined to be the optimal scheme for the synthesis of high-purity SAHA product suitable for GMP manufacturing and up-scaling to produce larger quantities.

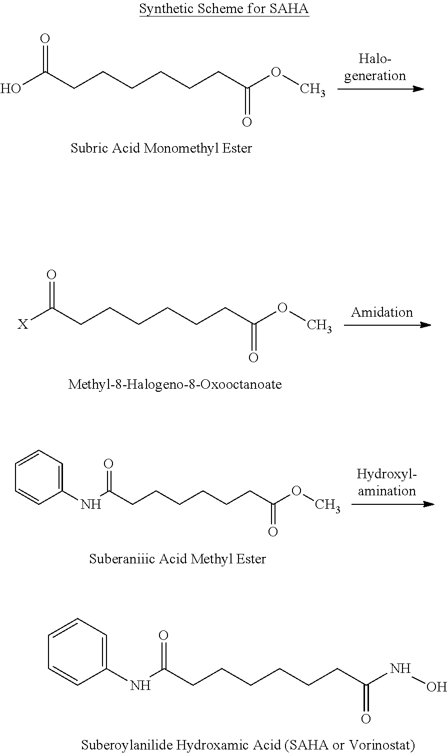

With the three-step synthetic scheme provided herein, SAHA can be prepared in a consecutive manner without purification of any intermediate, and advantageously in a purity of at least 90% with a three-step overall yield of at least 90%. The SAHA product produced by this method and having a purity of at least 90% with a three step overall yield of at least 90% is referred to as “high-purity” SAHA.

According to the present invention, the SAHA product directly obtained by the three-step synthetic scheme, without any purification, can have a purity as high as at least 90%, at least 91%, at least 92%, at least 93%, at least 94%, at least 95%, at least 96%, at least 97%, or at least 98%. Such high-purity SAHA product may be used for various purposes, or may be easily further purified by non-chromatographic purifications to achieve a higher purity of at least 99%, preferably at least 99.9%.

As used herein and unless otherwise specified, the term “non-chromatographic purification” refers to any process for purifying a compound including, but being not limited to, distillation, sublimation, recrystallization, precipitation, and extraction. In some embodiments, the SAHA product directly obtained by the three-step synthetic scheme is further purified by recrystallization to achieve a purity of at least 99%, preferably at least 99.9%.

According to the present invention, the SAHA product directly obtained by the three-step synthetic scheme, without any purification, can be obtained with a yield of at least 90%, at least 91%, at least 92%, at least 93%, at least 94%, at least 95%, at least 96%, at least 97%, at least 98%, or at least 99%.

After further non-chromatographic purification, the SAHA product with a purity of at least 99%, preferably at least 99.9%, can be obtained with a yield of at least 80%, at least 81%, at least 82%, at least 83%, at least 84%, at least 85%, at least 86%, at least 87%, at least 88%, at least 89%, or at least 90%, throughout the three steps of the synthetic scheme and the non-chromatographic purification provided herein. In some embodiments, after further non-chromatographic purifications, the SAHA product with a purity of at least 99%, preferably at least 99.9%, can reach a high yield of 80%-90% throughout the three steps of the synthetic scheme and the non-chromatographic purification provided herein.

Specific embodiments for each step of the synthetic scheme are elaborated as follows.

### Halogenation

As used herein and unless otherwise specified, the term “halogenation” refers to a reaction in which one or more halogens are introduced into a compound.

According to the present invention, the commercially available and low-cost suberic acid monomethyl ester is used as the starting material, whose carboxyl group is activated through halogenation for facilitating the following amidation reaction. In some embodiments, the carboxyl group of suberic acid monomethyl ester is activated by replacing its hydroxyl group —OH with a halide group —X, wherein X is a halogen.

According to the present invention, the halogenation of suberic acid monomethyl ester comprises replacing its hydroxyl group —OH with a halide group —X, wherein X is selected from F, Cl, Br, and I. Thus, the halogenation of suberic acid monomethyl ester can be carried out by reacting suberic acid monomethyl ester with one or more halogenation reagents.

In preferred embodiments, the halogenation of suberic acid monomethyl ester comprises replacing its hydroxyl group —OH with —Cl. Thus, the halogenation of suberic acid monomethyl ester can be carried out by reacting suberic acid monomethyl ester with one or more chlorinating reagents to give the chlorinated compound, i.e., methyl-8-halogeno-8-oxooctanoate. Advantageously, the preparation of the chlorinated compound, compared to brominated, fluorinated, or iodinated compound, is very clean, practical, and cheap, which is also sufficiently reactive to give high yield of amide during the following amidation process.

As used herein and unless otherwise specified, the term “halogenation reagent” refers to a chemical used to introduce halogens into a compound. The halogenation reagents suitable for use in the present invention include, but are not limited to, 1) fluorinating reagents, such as potassium hydrogenfluoride (KHF₂), cesium fluoride (CsF), tetramethylammonium fluoride tetrahydrate, tetrabutylammonium fluoride, triethylamine trihydrofluoride, IF₅-Pyridine-HF, and PyFluor; 2) chlorinating reagents, such as chlorine (Cl₂), thionyl chloride (SOCl₂), methanesulfonyl chloride, trichloromethanesulfonyl chloride, tert-butyl hypochlorite, dichloromethyl methyl ether, methoxyacetyl chloride, oxalyl chloride ((COCl)₂), phosphorus pentachloride (PCl₅), phosphorus trichloride (PCl₃), and trimethylsilyl chloride; 3) brominating reagents, such as bromine (Br₂), carbon tetrabromide (CBr₄), boron tribromide (BBr₃), phosphorus tribromide (PBr₃), bromotrichloromethane, 1,2-dibromo-1,1,2,2-tetrachloroethane, tetrabutylammonium tribromide, and trimethylsilyl bromide; and 4) iodinating reagents, such as iodine (I₂), hydriodic acid (HI), carbon tetraiodide (CI₄), trimethylsilyl iodide, 1-chloro-2-iodoethane, N,N-dimethyl-N-(methylsulfanylmethylene)-ammonium iodide, and pyridine iodine monochloride.

In some embodiments, the halogenation of suberic acid monomethyl ester is carried out by reacting suberic acid monomethyl ester with one or more chlorinating reagents, preferably with thionyl chloride (SOCl₂) or oxalyl chloride ((COCl)₂).

According to the present invention, the halogenation of suberic acid monomethyl ester can be carried out in a molar ratio of suberic acid monomethyl ester: halogenation reagent of about 1:(1-2), about 1:(1.05-1.9), about 1:(1.1-1.8), about 1:(1.15-1.7), about 1:(1.2-1.6), about 1:(1.25-1.5), or about 1:(1.3-1.4). In some embodiments, the halogenation of suberic acid monomethyl ester is carried out in a molar ratio of suberic acid monomethyl ester: halogenation reagent of about 1:(1.25-1.3).

According to the present invention, the halogenation of suberic acid monomethyl ester can be carried out in a solvent selected from benzene, toluene, xylene, ethylbenzene, mesitylene, o-dichlorobenzene, any other suitable solvents, and any mixture thereof. In a preferred embodiment, the halogenation of suberic acid monomethyl ester can be carried out in toluene, which offers advantageous properties compared to other solvents. In some embodiments, benzene is not used as a solvent in the halogenation reaction due to high toxicity. In some embodiments, other solvents such as xylene, ethylbenzene, mesitylene, o-dichlorobenzene, and any mixture thereof are not used in the halogenation reaction due to their high boiling points.

The solvents suitable for use in the halogenation of suberic acid monomethyl ester are preferably dry solvents. As used herein and unless otherwise specified, the term “dry solvent” refers to a solvent that contains up to about 5%, up to about 4%, up to about 3%, up to about 2%, up to about 1%, up to about 0.1%, up to about 0.01% water, or be substantially free of water, or be anhydrous. In some embodiments, the halogenation of suberic acid monomethyl ester is carried out in a solvent of dry toluene.

According to the present invention, the halogenation of suberic acid monomethyl ester can be carried out at a temperature of about 20-80° C., about 25-75° C., about 30-70° C., about 35-65° C., about 40-60° C., or about 45-55° C. In some embodiments, the halogenation of suberic acid monomethyl ester is carried out at a temperature of about 50-60° C., preferably about 52-58° C., more preferably about 54-56° C.

According to the present invention, the halogenation of suberic acid monomethyl ester can be carried out under an inert atmosphere of argon or nitrogen.

According to the present invention, the halogenation of suberic acid monomethyl ester can be carried out for about 5-30 hrs, about 7-28 hrs, about 9-26 hrs, about 11-24 hrs, about 13-22 hrs, about 15-20 hrs, or about 17-18 hrs, in order to allow a sufficient reaction. In some embodiments, the halogenation of suberic acid monomethyl ester is carried out for about 10-20 hrs, preferably about 12-18 hrs, more preferably about 14-16 hrs.

According to the present invention, the halogenation of suberic acid monomethyl ester can be carried out by reacting suberic acid monomethyl ester with one or more halogenation reagents in a molar ratio of suberic acid monomethyl ester: halogenation reagent of about 1:(1-2), in a solvent selected from toluene, any other suitable solvents, and any mixture thereof, at a temperature of about 20-80° C. and under an inert atmosphere of argon or nitrogen, for about 5-30 hrs.

In some embodiments, the halogenation of suberic acid monomethyl ester can be carried out by reacting suberic acid monomethyl ester with one or more chlorinating reagents in a molar ratio of suberic acid monomethyl ester: chlorinating reagent of about 1:(1.15-1.7), in a solvent of toluene, at a temperature of about 40-60° C. and under an inert atmosphere of argon or nitrogen, for about 10-20 hrs.

In preferred embodiments, the halogenation of suberic acid monomethyl ester can be carried out by reacting suberic acid monomethyl ester with thionyl chloride (SOCl₂) in a molar ratio of suberic acid monomethyl ester: thionyl chloride of about 1:(1.25-1.3), in a solvent of dry toluene, at a temperature of about 54-56° C. and under an inert atmosphere of argon, for about 14-16 hrs.

The present invention aims to simplify the production of SAHA by eliminating purification of intermediates, especially by eliminating chromatographic purification of intermediates. In some embodiments, the crude methyl-8-halogeno-8-oxooctanoate obtained from the halogenation of suberic acid monomethyl ester is directly used in following reactions without chromatographic purification or non-chromatographic purification. In other embodiments, the crude methyl-8-halogeno-8-oxooctanoate obtained from the halogenation of suberic acid monomethyl ester is used in following reactions after chromatographic purification and/or non-chromatographic purification.

### Amidation

As used herein and unless otherwise specified, the term “amidation” refers to a reaction in which an amide group is created.

Advantageously, the crude methyl-8-halogeno-8-oxooctanoate obtained from the halogenation of suberic acid monomethyl ester can be directly used in the amidation reaction without chromatographic purification or non-chromatographic purification. In some embodiments, the crude methyl-8-halogeno-8-oxooctanoate obtained from the halogenation of suberic acid monomethyl ester is directly used in the amidation reaction after removing volatiles, including, but being not limited to, the solvents used in the halogenation reaction.

According to the present invention, the amidation of methyl-8-halogeno-8-oxooctanoate comprises replacing its halide group (—X, wherein X is a halogen) with an anilino group (—NH—C₆H₅).

According to the present invention, the amidation of methyl-8-halogeno-8-oxooctanoate can be carried out by reacting methyl-8-halogeno-8-oxooctanoate with aniline, preferably in the presence of one or more alkaline catalysts.

During amidation, the alkaline catalysts serve as bases to neutralize haloid acid generated during the reaction, i.e., to remove free haloid acid, such as hydrogen chloride, from the reaction mixture by forming a salt, and thus shifting the equilibrium towards the forward direction and favoring amide group formation. In some embodiments, the alkaline catalysts can be first mixed with aniline before being added to methyl-8-halogeno-8-oxooctanoate, in order to sufficiently deprotonate aniline. In other embodiments, the alkaline catalysts, aniline, and methyl-8-halogeno-8-oxooctanoate are mixed together simultaneously.

The alkaline catalysts suitable for use in the present invention include, but are not limited to, 1) organic alkaline catalysts, such as pyridine, morpholine, triethylamine (TEA), dimethylaniline, guanidine, 1,5,7-triazabicyclodec-5-ene (TBD), 1,8-diazabicyclo[5.4.0]undec-7-ene (DBU), hexamethylphosphoric triamide (HMPA), imidazole, and acetamide; and 2) inorganic alkaline catalysts, such as calcium oxide, magnesium oxide, sodium hydroxide, calcium hydroxide, potassium hydroxide, potassium carbonate, nickel hydroxide, and zinc hydroxide.

In some embodiments, the amidation of methyl-8-halogeno-8-oxooctanoate is carried out by reacting methyl-8-halogeno-8-oxooctanoate with aniline in the presence of pyridine or TEA. In preferred embodiments, TEA is much superior as an alkaline catalyst in the amidation reaction because it is cheap, available as anhydrous reagent, and forms triethylamine HCl salt, which is almost insoluble in THF and therefore can easily be removed by simple filtration.

According to the present invention, the amidation of methyl-8-halogeno-8-oxooctanoate can be carried out in a molar ratio of theoretical methyl-8-halogeno-8-oxooctanoate: aniline: alkaline catalyst of about 1:(1-2):(1-2), about 1:(1-1.8):(1-1.8), about 1:(1-1.6):(1-1.6), about 1:(1-1.4):(1-1.4), about 1:(1-1.2):(1-1.2), about 1:(1-1.1):(1-1.1), or about 1:(1-1.05):(1-1.05).

As used herein and unless otherwise specified, the term “theoretical”, when used in connection with a compound, means the theoretical amount or theoretical yield of such compound resulting from a previous process or reaction at 100% reactant conversion. For example, “theoretical methyl-8-halogeno-8-oxooctanoate” refers to the theoretical amount of methyl-8-halogeno-8-oxooctanoate obtained from the previous halogenation reaction at 100% suberic acid monomethyl ester conversion. This means that “theoretical methyl-8-halogeno-8-oxooctanoate” is 1 mol when 1 mol of suberic acid monomethyl ester is used in the halogenation reaction. “Theoretical suberanilic acid methyl ester” refers to the theoretical amount of suberanilic acid methyl ester obtained from the previous amidation reaction at 100% suberic acid monomethyl ester conversion and 100% methyl-8-halogeno-8-oxooctanoate conversion, which means that “theoretical suberanilic acid methyl ester” is 1 mol when 1 mol of suberic acid monomethyl ester is used in the halogenation reaction.

According to the present invention, the amidation of methyl-8-halogeno-8-oxooctanoate can be carried out in a solvent selected from tetrahydrofuran (THF), 1,4-dioxane, gamma-valerolactone (GVL), 2-mehtoxyethanol (MOE), dimethyl sulfoxide (DMSO), dimethylformamide (DMF), any other suitable solvents, and any mixture thereof. In a preferred embodiment, the amidation of methyl-8-halogeno-8-oxooctanoate is carried out in THF. In some embodiments, other solvents such as 1,4-dioxane, gamma-valerolactone (GVL), 2-mehtoxyethanol (MOE), dimethyl sulfoxide (DMSO), dimethylformamide (DMF), and any mixture thereof are not used in the amidation reaction because they will not work at all and give nightmarish situation as far as the work up and subsequent purification are concerned, and none of them offers any advantage over tetrahydrofuran (THF) to the present invention. The solvents suitable for use in the amidation of methyl-8-halogeno-8-oxooctanoate are preferably dry solvents. In some embodiments, the amidation of methyl-8-halogeno-8-oxooctanoate is carried out in a solvent of dry tetrahydrofuran.

According to the present invention, the amidation of methyl-8-halogeno-8-oxooctanoate can be carried out by mixing the alkaline catalysts, aniline, and methyl-8-halogeno-8-oxooctanoate at a temperature of about 0-10° C., about 1-9° C., about 2-8° C., about 3-7° C., about 4-6° C., about 0-5° C., about 0-4° C., about 0-3° C., about 0-2° C., or about 0-1° C., and then reacting at a temperature of about 10-30° C., about 12-28° C., about 14-26° C., about 16-24° C., or about 18-22° C. In some embodiments, the amidation of methyl-8-halogeno-8-oxooctanoate is carried out by mixing the alkaline catalysts, aniline, and methyl-8-halogeno-8-oxooctanoate at a temperature of about 0-5° C., and then reacting at a temperature of about 20-25° C.

According to the present invention, the amidation of methyl-8-halogeno-8-oxooctanoate can be carried out under an inert atmosphere of argon or nitrogen.

According to the present invention, the amidation of methyl-8-halogeno-8-oxooctanoate can be carried out for about 5-30 hrs, about 7-28 hrs, about 9-26 hrs, about 11-24 hrs, about 13-22 hrs, about 15-20 hrs, or about 17-18 hrs, in order to allow a sufficient reaction. In some embodiments, the amidation of methyl-8-halogeno-8-oxooctanoate is carried out for about 10-20 hrs, preferably about 12-18 hrs, more preferably about 14-16 hrs.

According to the present invention, the amidation of methyl-8-halogeno-8-oxooctanoate can be carried out by reacting methyl-8-halogeno-8-oxooctanoate with aniline in the presence of one or more alkaline catalysts in a molar ratio of theoretical methyl-8-halogeno-8-oxooctanoate: aniline: alkaline catalyst of about 1:(1-2):(1-2), in a solvent selected from tetrahydrofuran (THF), any other suitable solvents, and any mixture thereof, at a temperature of about 10-30° C. and under an inert atmosphere of argon or nitrogen, for about 5-30 hrs.

In some embodiments, the amidation of methyl-8-halogeno-8-oxooctanoate is carried out by under an inert atmosphere of argon or nitrogen and in a solvent of tetrahydrofuran, mixing the alkaline catalysts, aniline, and methyl-8-halogeno-8-oxooctanoate at a temperature of about 0-10° C. at a molar ratio of theoretical methyl-8-halogeno-8-oxooctanoate: aniline: alkaline catalyst of about 1:(1-2):(1-2), and then reacting at a temperature of about 10-30° C. for about 5-30 hrs.

In preferred embodiments, the amidation of methyl-8-halogeno-8-oxooctanoate is carried out by under an inert atmosphere of argon and in a solvent of tetrahydrofuran, at a temperature of about 0-10° C., firstly mixing the alkaline catalysts with aniline, followed by adding the mixture of the alkaline catalysts and aniline into methyl-8-halogeno-8-oxooctanoate, at a molar ratio of theoretical methyl-8-halogeno-8-oxooctanoate: aniline: alkaline catalyst of about 1:(1-2):(1-2), and then reacting at a temperature of about 10-30° C. for about 5-30 hrs.

In more preferred embodiments, the amidation of methyl-8-halogeno-8-oxooctanoate is carried out by under an inert atmosphere of argon and in a solvent of dry tetrahydrofuran, at a temperature of about 0-5° C., firstly mixing triethylamine with aniline, followed by adding the mixture of triethylamine and aniline into methyl-8-halogeno-8-oxooctanoate, at a molar ratio of theoretical methyl-8-halogeno-8-oxooctanoate: aniline: triethylamine of about 1:(1-1.05):(1-1.05), and then reacting at a temperature of about 20-25° C. for about 10-20 hrs.

In some embodiments, the crude suberanilic acid methyl ester obtained from the amidation of methyl-8-halogeno-8-oxooctanoate is directly used in following reactions without chromatographic purification or non-chromatographic purification. In other embodiments, the crude suberanilic acid methyl ester obtained from the amidation of methyl-8-halogeno-8-oxooctanoate is used in following reactions after chromatographic purification and/or non-chromatographic purification, such as, after filtration to remove solid by-products.

### Hydroxylamination

As used herein and unless otherwise specified, the term “hydroxylamination” refers to a reaction in which one or more hydroxylamino groups (—NH—OH) are introduced into a compound.

Advantageously, the crude suberanilic acid methyl ester obtained from the amidation of methyl-8-halogeno-8-oxooctanoate can be directly used in the hydroxylamination reaction without chromatographic purification or non-chromatographic purification. In some embodiments, the crude suberanilic acid methyl ester obtained from the amidation of methyl-8-halogeno-8-oxooctanoate is directly used in the hydroxylamination reaction after removing volatiles, including, but being not limited to, the solvents used in the amidation reaction.

According to the present invention, the hydroxylamination of suberanilic acid methyl ester comprises replacing its methoxy group (—O—CH₃) with a hydroxylamino group (—NH—OH).

According to the present invention, the hydroxylamination of suberanilic acid methyl ester can be carried out by reacting suberanilic acid methyl ester with hydroxylamine, preferably in the presence of one or more alkaline catalysts.

Hydroxylamine used in the present invention can be in the form of hydroxylamine salts, including, but being not limited to, hydroxylamine hydrochloride, hydroxylamine sulfate, sodium hydroxylamine, and potassium hydroxylamine.

In some embodiments, the alkaline catalysts can be firstly mixed with hydroxylamine before being added to suberanilic acid methyl ester, in order to sufficiently deprotonate hydroxylamine. In other embodiments, the alkaline catalysts, hydroxylamine, and suberanilic acid methyl ester are mixed together almost simultaneously.

The alkaline catalysts suitable for use in the present invention include, but are not limited to, sodium hydroxide (NaOH), potassium hydroxide (KOH), calcium hydroxide (Ca(OH)₂), sodium carbonate (Na₂CO₃), potassium carbonate (K₂CO₃), sodium bicarbonate (NaHCO₃), ammonia (NH₃), sodium methylate/sodium methoxide (CH₃ONa), sodium ethoxide (C₂H₅ONa), potassium tert-butoxide ((CH₃)₃COK), 1,5,7-triazabicyclo[4.4.0]dec-5-ene (TBD), and 1,8-diazabicyclo[5.4.0]undec-7-ene (DBU).

In some embodiments, the hydroxylamination of suberanilic acid methyl ester is carried out by reacting suberanilic acid methyl ester with hydroxylamine in the presence of sodium methylate or sodium ethoxide.

According to the present invention, the hydroxylamination of suberanilic acid methyl ester can be carried out at a molar ratio of theoretical suberanilic acid methyl ester: hydroxylamine: alkaline catalyst of about 1:(1-10):(1-10), about 1:(2-9):(2-9), about 1:(3-8):(3-8), about 1:(4-7):(4-7), about 1:(4-6):(4-6), or about 1:5:(5-5.5).

According to the present invention, the hydroxylamination of suberanilic acid methyl ester can be carried out in a solvent selected from methanol, ethanol, isopropanol, dimethyl sulfoxide (DMSO), dimethylformamide (DMF), acetonitrile, any other suitable solvents, and any mixture thereof. In some embodiments, other solvents such as ethanol, isopropanol, dimethyl sulfoxide (DMSO), dimethylformamide (DMF), acetonitrile, and any mixture thereof are not used in the hydroxylamination reaction because they will not work at all or are impractical to use or will generate large amount of side product, and none of them offers any advantage over methanol to the present invention. In some embodiments, the solvents for use in the hydroxylamination of suberanilic acid methyl ester are dry solvents for avoiding water in the reaction mixture. In preferred embodiments, the hydroxylamination of suberanilic acid methyl ester is carried out in a solvent of dry methanol.

In other embodiments, the solvents for use in the hydroxylamination of suberanilic acid methyl ester can contain at least about 10%, at least about 20%, at least about 30%, at least about 40%, at least about 50%, at least about 60%, at least about 70%, at least about 80%, or at least about 90% water, or be water.

According to the present invention, the hydroxylamination of suberanilic acid methyl ester can be carried out by firstly mixing the alkaline catalysts with hydroxylamine at a temperature of about 0-10° C., about 1-9° C., about 2-8° C., about 3-7° C., about 4-6° C., about 0-5° C., about 0-4° C., about 0-3° C., about 0-2° C., or about 0-1° C., followed by adding suberanilic acid methyl ester into the mixture of the alkaline catalysts and hydroxylamine at a temperature of about 10-20° C., about 11-19° C., about 12-18° C., about 13-17° C., or about 14-16° C., and then reacting at a temperature of about 10-30° C., about 12-28° C., about 14-26° C., about 16-24° C., or about 18-22° C. In some embodiments, the hydroxylamination of suberanilic acid methyl ester is carried out by firstly mixing the alkaline catalysts with hydroxylamine at a temperature of about 0-5° C., followed by adding suberanilic acid methyl ester into the mixture of the alkaline catalysts and hydroxylamine at a temperature of about 14-16° C., and then reacting at a temperature of about 20-25° C.

According to the present invention, the hydroxylamination of suberanilic acid methyl ester can be carried out under an inert atmosphere of argon or nitrogen.

According to the present invention, the hydroxylamination of suberanilic acid methyl ester can be carried out for about 5-30 hrs, about 7-28 hrs, about 9-26 hrs, about 11-24 hrs, about 13-22 hrs, about 15-20 hrs, or about 17-18 hrs, in order to allow a sufficient reaction. In some embodiments, the hydroxylamination of suberanilic acid methyl ester is carried out for about 10-20 hrs, preferably about 12-18 hrs, more preferably about 14-16 hrs.

According to the present invention, the hydroxylamination of suberanilic acid methyl ester can be carried out by reacting suberanilic acid methyl ester with hydroxylamine under existence of one or more alkaline catalysts in a molar ratio of theoretical suberanilic acid methyl ester: hydroxylamine: alkaline catalyst of about 1:(1-10):(1-10), in a solvent selected from methanol, any other suitable solvents, and any mixture thereof, at a temperature of about 10-30° C. and under an inert atmosphere of argon or nitrogen, for about 5-30 hrs.

In some embodiments, the hydroxylamination of suberanilic acid methyl ester is carried out by under an inert atmosphere of argon or nitrogen and in a solvent of methanol, firstly mixing the alkaline catalysts with hydroxylamine at a temperature of about 0-10° C., followed by adding suberanilic acid methyl ester into the mixture of the alkaline catalysts and hydroxylamine at a temperature of about 10-20° C., in a molar ratio of theoretical suberanilic acid methyl ester: hydroxylamine: alkaline catalyst of about 1:(1-10):(1-10), and then reacting at a temperature of about 10-30° C. for about 5-30 hrs.

In preferred embodiments, the hydroxylamination of suberanilic acid methyl ester is carried out by under an inert atmosphere of argon and in a solvent of dry methanol, firstly mixing sodium methylate with hydroxylamine hydrochloride at a temperature of about 0-10° C., followed by adding suberanilic acid methyl ester into the mixture of sodium methylate and hydroxylamine hydrochloride at a temperature of about 10-20° C., in a molar ratio of theoretical suberanilic acid methyl ester: hydroxylamine hydrochloride: sodium methylate of about 1:(1-10):(1-10), and then reacting at a temperature of about 10-30° C. for about 5-30 hrs.

In more preferred embodiments, the hydroxylamination of suberanilic acid methyl ester is carried out by under an inert atmosphere of argon and in a solvent of dry methanol, firstly mixing sodium methylate with hydroxylamine hydrochloride at a temperature of about 0-5° C., followed by adding suberanilic acid methyl ester into the mixture of sodium methylate and hydroxylamine hydrochloride at a temperature of about 14-16° C., in a molar ratio of theoretical suberanilic acid methyl ester: hydroxylamine hydrochloride: sodium methylate of about 1:(1-10):(1-10), and then reacting at a temperature of about 20-25° C. for about 10-20 hrs.

According to the present invention, upon cease of the hydroxylamination reaction, the reaction mixture can be mixed with cold water to facilitate precipitation of the final product SAHA. The cold water suitable for use in the present invention can have a temperature of about 0-5° C., about 0-4° C., about 0-3° C., about 0-2° C., or about 0-1° C. The volume ratio of the reaction mixture: cold water can be about 1:(1.5-3), about 1:(1.6-2.9), about 1:(1.7-2.8), about 1:(1.8-2.7), about 1:(1.9-2.6), about 1:(2-2.5), about 1:(2.1-2.4), or about 1:(2.2-2.4).

According to the present invention, the precipitated SAHA can be washed with solvents selected from water, ethyl acetate, tetrahydrofuran, and any mixture thereof. In some embodiments, the precipitated SAHA is washed with water, followed by washed with ethyl acetate. In some embodiments, the precipitated SAHA is washed with water, followed by washed with tetrahydrofuran.

According to the present invention, the precipitated SAHA, after washing but without any purification, can have a purity as high as at least 90%, at least 91%, at least 92%, at least 93%, at least 94%, at least 95%, at least 96%, at least 97%, or at least 98%.

According to the present invention, the precipitated SAHA, after washing but without any purification, can be yielded with at least 90%, at least 91%, at least 92%, at least 93%, at least 94%, at least 95%, at least 96%, at least 97%, at least 98%, or at least 99%, throughout the three steps, i.e., halogenation, amidation, and hydroxylamination, of the synthetic scheme provided herein.

### Non-Chromatographic Purification

While the present invention intends to eliminate chromatographic purification in preparation of SAHA, the precipitated SAHA can optionally go through non-chromatographic purification to achieve a higher purity of at least 99%, preferably at least 99.9%. The non-chromatographic purification suitable for use in the present invention includes, but is not limited to, distillation, sublimation, recrystallization, precipitation, and extraction.

In some embodiments, the precipitated SAHA can optionally go through a recrystallization from a mixture of methanol and ethyl acetate, wherein the ratio of theoretical SAHA: methanol: ethyl acetate (g: ml: ml) is about 1:(1-10):(1-10), about 1:(2-9):(2-9), about 1:(3-8):(3-8), about 1:(4-7):(4-7), or about 1:(5-7):(5-7).

In some embodiments, the precipitated SAHA can optionally go through a recrystallization from a mixture of methanol and acetonitrile, wherein the ratio of theoretical SAHA: methanol: acetonitrile (g: ml: ml) is about 1:(1-10):(1-10), about 1:(2-9):(2-9), about 1:(3-8):(3-8), about 1:(4-7):(4-7), or about 1:(5-7):(5-7).

In some embodiments, the recrystallized SAHA can be optionally washed with solvents selected from ethyl acetate, and a mixture of ethyl acetate and methanol. The mixture of ethyl acetate and methanol suitable for use in the present invention can have a volume ratio of ethyl acetate: methanol of about 9:1, about 8:2, about 7:3, about 6:4, or about 5:5. In preferred embodiments, the recrystallized SAHA is washed with a mixture of ethyl acetate and methanol in volume ratio of ethyl acetate: methanol of about 8:2, followed by washed with ethyl acetate.

In some embodiments, the recrystallized SAHA can be optionally washed with solvents selected from acetonitrile, and a mixture of acetonitrile and methanol. The mixture of acetonitrile and methanol suitable for use in the present invention can have a volume ratio of acetonitrile: methanol of about 9:1, about 8:2, about 7:3, about 6:4, or about 5:5. In preferred embodiments, the recrystallized SAHA is washed with a mixture of acetonitrile and methanol in volume ratio of acetonitrile: methanol of about 8:2, followed by washed with acetonitrile.

In some embodiments, the recrystallized SAHA, after washing but without any chromatographic purification, can have a purity as high as at least 99%, or at least 99.9%.

In some embodiments, the recrystallized SAHA, after washing but without any chromatographic purification, can be yielded with at least 80%, at least 81%, at least 82%, at least 83%, at least 84%, at least 85%, at least 86%, at least 87%, at least 88%, at least 89%, or at least 90%, throughout the three steps, i.e., halogenation, amidation, and hydroxylamination, of the synthetic scheme provided herein.

### Strategies for Preparing SAHA and Precursors Thereof

With reference to the three-step synthetic scheme and embodiments described herein, the present invention intends to provide various strategies for preparing SAHA and SAHA precursors in both high purity and high yield.

In one embodiment, provided herein is a method for preparing SAHA from suberic acid monomethyl ester, the method comprising steps of:
(a) halogenation of suberic acid monomethyl ester as described herein, to obtain methyl-8-halogeno-8-oxooctanoate;
(b) amidation of methyl-8-halogeno-8-oxooctanoate as described herein, to obtain suberanilic acid methyl ester;
(c) hydroxylamination of suberanilic acid methyl ester as described herein, to obtain SAHA; and
(d) optionally non-chromatographic purification of SAHA, to obtain SAHA with a purity of at least 99%.

In some embodiments, step (a) of the method is carried out by reacting suberic acid monomethyl ester with a halogenation reagent at a molar ratio of suberic acid monomethyl ester: halogenation reagent of about 1:(1-2), and/or step (a) of the method is carried out at a temperature of about 20-60° C.

In specific embodiments, the halogenation reagent is selected from chlorine (Cl₂), thionyl chloride (SOCl₂), methanesulfonyl chloride, trichloromethanesulfonyl chloride, tert-butyl hypochlorite, dichloromethyl methyl ether, methoxyacetyl chloride, oxalyl chloride ((COCl)₂), phosphorus pentachloride (PCl₅), phosphorus trichloride (PCl₃), and trimethylsilyl chloride. In a specific embodiment, the halogenation reagent is SOCl₂.

In some embodiments, step (b) of the method is carried out by reacting methyl-8-halogeno-8-oxooctanoate with aniline in the presence of a first alkaline catalyst at a molar ratio of methyl-8-halogeno-8-oxooctanoate: aniline: first alkaline catalyst of about 1:(1-2):(1-2), and/or step (b) of the method is carried out by mixing the first alkaline catalyst with aniline, followed by adding the mixture of the first alkaline catalyst and aniline into methyl-8-halogeno-8-oxooctanoate. In a specific embodiment, the first alkaline catalyst is triethylamine.

In some embodiments, step (c) of the method is carried out by reacting suberanilic acid methyl ester with hydroxylamine in the presence of a second alkaline catalyst at a molar ratio of suberanilic acid methyl ester: hydroxylamine: second alkaline catalyst of about 1:(1-10):(1-10), and/or step (c) of the method is carried out by mixing the second alkaline catalyst with hydroxylamine, followed by adding suberanilic acid methyl ester into the mixture of the second alkaline catalyst and hydroxylamine. In a specific embodiment, the second alkaline catalyst is sodium methylate.

In one embodiment, provided herein is a method for preparing SAHA from methyl-8-halogeno-8-oxooctanoate, the method comprising steps of:
(a) amidation of methyl-8-halogeno-8-oxooctanoate as described herein, to obtain suberanilic acid methyl ester;
(b) hydroxylamination of suberanilic acid methyl ester as described herein, to obtain SAHA; and
(c) optionally non-chromatographic purification of SAHA, to obtain SAHA with a purity of at least 99%.

In some embodiments, methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

In some embodiments, step (a) of the method is carried out by reacting methyl-8-halogeno-8-oxooctanoate with aniline in the presence of a first alkaline catalyst at a molar ratio of methyl-8-halogeno-8-oxooctanoate: aniline: first alkaline catalyst of about 1:(1-2):(1-2). In a specific embodiment, the first alkaline catalyst is triethylamine.

In some embodiments, step (b) of the method is carried out by reacting suberanilic acid methyl ester with hydroxylamine in the presence of a second alkaline catalyst at a molar ratio of theoretical suberanilic acid methyl ester: hydroxylamine: second alkaline catalyst of about 1:(1-10):(1-10). In a specific embodiment, the second alkaline catalyst is sodium methylate.

In one embodiment, provided herein is a method for preparing SAHA from suberanilic acid methyl ester, the method comprising steps of:
(a) hydroxylamination of suberanilic acid methyl ester as described herein, to obtain SAHA; and
(b) optionally non-chromatographic purification of SAHA, to obtain SAHA with a purity of at least 99%.

In certain embodiments, step (a) is carried out by reacting suberanilic acid methyl ester with hydroxylamine in the presence of an alkaline catalyst at a molar ratio of suberanilic acid methyl ester: hydroxylamine: alkaline catalyst of about 1:(1-10):(1-10). In certain embodiments, step (a) is carried out by mixing the alkaline catalyst with hydroxylamine, followed by adding suberanilic acid methyl ester into the mixture of the alkaline catalyst and hydroxylamine. In a specific embodiment, the alkaline catalyst is sodium methylate.

In one embodiment, provided herein is a method for preparing suberanilic acid methyl ester, which is a precursor of SAHA, from suberic acid monomethyl ester, the method comprising steps of:
(a) halogenation of suberic acid monomethyl ester as described herein, to obtain methyl-8-halogeno-8-oxooctanoate; and
(b) amidation of methyl-8-halogeno-8-oxooctanoate as described herein, to obtain suberanilic acid methyl ester.

In certain embodiments, step (a) is carried out by reacting suberic acid monomethyl ester with a halogenation reagent at a molar ratio of suberic acid monomethyl ester: halogenation reagent of about 1:(1-2). In a preferred embodiment, step (a) is carried out at a temperature of about 20-80° C.

In specific embodiments, the halogenation reagent is selected from chlorine (Cl₂), thionyl chloride (SOCl₂), methanesulfonyl chloride, trichloromethanesulfonyl chloride, tert-butyl hypochlorite, dichloromethyl methyl ether, methoxyacetyl chloride, oxalyl chloride ((COCl)₂), phosphorus pentachloride (PCl₅), phosphorus trichloride (PCl₃), and trimethylsilyl chloride. In a preferred embodiment, the halogenation reagent is SOCl₂.

In certain embodiments, step (b) is carried out by reacting methyl-8-halogeno-8-oxooctanoate with aniline in the presence of an alkaline catalyst at a molar ratio of theoretical methyl-8-halogeno-8-oxooctanoate: aniline: alkaline catalyst of about 1:(1-2):(1-2). In certain embodiments, step (b) is carried out by mixing the alkaline catalyst with aniline, followed by adding the mixture of the alkaline catalyst and aniline into methyl-8-halogeno-8-oxooctanoate. In a specific embodiment, the alkaline catalyst is triethylamine.

In one embodiment, provided herein is a method for preparing suberanilic acid methyl ester, which is a precursor of SAHA, from methyl-8-halogeno-8-oxooctanoate, the method comprising a step of:
amidation of methyl-8-halogeno-8-oxooctanoate as described herein, to obtain suberanilic acid methyl ester.

In one embodiment, the subject invention provides a method for synthesizing high-purity SAHA, the method comprising:
mixing suberic acid monomethyl ester with a halogenation reagent (e.g., SOCl₂) to obtain methyl-8-halogeno-8-oxooctanoate;
reacting methyl-8-halogeno-8-oxooctanoate with aniline in the presence of a first alkaline catalyst (e.g., triethylamine) to obtain suberanilic acid methyl ester;
reacting suberanilic acid methyl ester with hydroxylamine in the presence of a second alkaline catalyst (e.g., sodium methylate) to obtain SAHA (e.g., at least 90% pure); and
optionally, purifying the obtained SAHA via, for example, non-chromatographic purification to obtain high-purity SAHA (e.g., at least 99% pure).

In certain embodiments, the reaction of methyl-8-halogeno-8-oxooctanoate with aniline in the presence of the first alkaline catalyst is carried out by mixing the first alkaline catalyst with aniline, followed by adding the mixture of the first alkaline catalyst and aniline into methyl-8-halogeno-8-oxooctanoate.

In certain embodiments, the reaction of suberanilic acid methyl ester with hydroxylamine in the presence of the second alkaline catalyst is carried out by mixing the second alkaline catalyst with hydroxylamine, followed by adding suberanilic acid methyl ester into the mixture of the second alkaline catalyst and hydroxylamine.

In one embodiment, the subject invention provides a method for synthesizing high-purity SAHA, the method comprising:
(a) mixing suberic acid monomethyl ester with a halogenation reagent (e.g., SOCl₂) to obtain methyl-8-halogeno-8-oxooctanoate;
(b) adding a mixture of aniline and a first alkaline catalyst (e.g., triethylamine) into (a) to obtain suberanilic acid methyl ester;
(c) adding suberanilic acid methyl ester into a mixture of a second alkaline catalyst (e.g., sodium methylate) and hydroxylamine to obtain SAHA (e.g., at least 90% pure); and
(d) optionally, purifying the obtained SAHA of step (c), via, for example, non-chromatographic purification, to obtain high-purity SAHA (e.g., at least 99% pure).

In one embodiment, the subject invention provides a method for synthesizing high-purity SAHA, the method comprising:
(a) adding a mixture of aniline and a first alkaline catalyst (e.g., triethylamine) into methyl-8-halogeno-8-oxooctanoate to obtain suberanilic acid methyl ester;
(b) adding suberanilic acid methyl ester into a mixture of a second alkaline catalyst (e.g., sodium methylate) and hydroxylamine to obtain SAHA (e.g., at least 90% pure); and
(c) optionally, purifying the obtained SAHA of step (b), via, for example, non-chromatographic purification, to obtain high-purity SAHA (e.g., at least 99% pure).

In certain embodiments, each reaction step may or may not be occurred in a solvent. In certain embodiments, if a solvent is used, the solvent may be removed at the end of the step before a different solvent is used in the next step.

In certain embodiments, in all the methods for preparing SAHA or SAHA precursors provided by the present invention, the methods comprise no chromatographic purification or non-chromatographic purification of intermediate obtained in each step.

In certain embodiments, in all the methods for preparing SAHA or SAHA precursors provided by the present invention, the reaction of each step is carried out in a dry solvent that contains up to about 5%, up to about 4%, up to about 3%, up to about 2%, up to about 1%, up to about 0.1%, up to about 0.01% water, or be substantially free of water, or be anhydrous.

In certain embodiments, in all the methods for preparing SAHA or SAHA precursors provided by the present invention, the reaction of each step is carried out under an inert atmosphere of argon or nitrogen.

In certain embodiments, in all the methods for preparing SAHA or SAHA precursors provided by the present invention, methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

In certain embodiments, the high-purity SAHA obtained from the synthesis processes of the subject invention can be used directly as a drug substance, for example, for treating cancer, and also be formulated directly as a pharmaceutical composition. Pharmaceutically acceptable excipients for such pharmaceutical compositions include standard carriers such as pharmaceutical grades of mannitol, lactose, starch, magnesium stearate, sodium saccharine, cellulose, microcrystalline cellulose, microcrystalline cellulose, sodium croscarmellose and magnesium stearate, sodium laurel sulfate, magnesium carbonate, etc. Other examples of suitable pharmaceutical carriers are described in “Remington's Pharmaceutical Sciences” by E. W. Martin. Such compositions will contain a therapeutically effective amount of the high-purity SAHA together with a suitable amount of carrier so as to provide the form for proper administration to the subject. The terms “carrier” and “excipient” can be used interchangeably herein.

Pharmaceutical compositions can comprise high-purity SAHA in admixture with a suitable carrier (excipient). Pharmaceutically acceptable carriers (excipients) include diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants. Non-limiting examples of non-aqueous pharmaceutically acceptable carriers (excipients) include propylene glycol, polyethylene glycol, vegetable oils such as olive oil, and injectable organic esters such as ethyl oleate. Pharmaceutically acceptable aqueous carriers (excipients) include water, alcoholic/aqueous solutions, emulsions or suspensions, including saline and buffered media. Parenteral vehicles (excipients/carriers) include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980, which is incorporated herein by reference. In some embodiments, high-purity SAHA can be formulated in commercially available oral syrup vehicles, such as ORA-SWEET, ORA-SWEET SF, ORA-PLUS, ORA-BLEND SF, or ORA-BLEND that are available from Padagis LLC (Piedmont, SC 29673). Thus, a pharmaceutical composition can be in liquid form or in the form of a solid or powder.

Compositions can be suitable for parenteral administration. Exemplary compositions are suitable for injection or infusion into an animal or human by any route available, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

When ranges are used herein, such as for dose ranges, combinations and subcombinations of ranges (e.g., subranges within the disclosed range), specific embodiments therein are intended to be explicitly included.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, or Z; X and Y; X and Z; Y and Z; or X, Y, and Z (i.e., any combination of X, Y, and Z).

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

This disclosure also includes the following additional claimable subject matter:

1. A method for preparing suberoylanilide hydroxamic acid (SAHA) from suberic acid monomethyl ester, comprising steps of:
(a) halogenation of suberic acid monomethyl ester, to obtain methyl-8-halogeno-8-oxooctanoate;
(b) amidation of methyl-8-halogeno-8-oxooctanoate, to obtain suberanilic acid methyl ester; and
(c) hydroxylamination of suberanilic acid methyl ester, to obtain SAHA.

2. The method of embodiment 1, further comprising a step of:
(d) non-chromatographic purification of SAHA.

3. The method of embodiment 1, wherein step (a) is carried out by reacting suberic acid monomethyl ester with a halogenation reagent at a molar ratio of suberic acid monomethyl ester: halogenation reagent of about 1:(1-2).

4. The method of embodiment 3, wherein the halogenation reagent is selected from chlorine (Cl₂), thionyl chloride (SOCl₂), methanesulfonyl chloride, trichloromethanesulfonyl chloride, tert-butyl hypochlorite, dichloromethyl methyl ether, methoxyacetyl chloride, oxalyl chloride ((COCl)₂), phosphorus pentachloride (PCl₅), phosphorus trichloride (PCl₃), and trimethylsilyl chloride.

5 The method of embodiment 4, wherein the halogenation reagent is SOCl₂.

6. The method of embodiment 1, wherein step (a) is carried out at a temperature of about 20-60° C.

7. The method of embodiment 1, wherein step (b) is carried out by reacting methyl-8-halogeno-8-oxooctanoate with aniline in the presence of a first alkaline catalyst at a molar ratio of methyl-8-halogeno-8-oxooctanoate: aniline: first alkaline catalyst of about 1:(1-2):(1-2).

8 The method of embodiment 7, wherein step (b) is carried out by firstly mixing the first alkaline catalyst with aniline, followed by adding the mixture of the first alkaline catalyst and aniline into methyl-8-halogeno-8-oxooctanoate.

9 The method of embodiment 7, wherein the first alkaline catalyst is triethylamine.

10. The method of embodiment 1, wherein step (c) is carried out by reacting suberanilic acid methyl ester with hydroxylamine in the presence of a second alkaline catalyst at a molar ratio of suberanilic acid methyl ester: hydroxylamine: second alkaline catalyst of about 1:(1-10):(1-10).

11. The method of embodiment 10, wherein step (c) is carried out by firstly mixing the second alkaline catalyst with hydroxylamine, followed by adding suberanilic acid methyl ester into the mixture of the second alkaline catalyst and hydroxylamine.

12. The method of embodiment 10, wherein the second alkaline catalyst is sodium methylate.

13. The method of embodiment 1, wherein step (a) and step (b) comprise no chromatographic purification or non-chromatographic purification of intermediate obtained in each step.

14. The method of embodiment 1, wherein methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

15. A method for preparing SAHA from methyl-8-halogeno-8-oxooctanoate, comprising steps of:
(a) amidation of methyl-8-halogeno-8-oxooctanoate, to obtain suberanilic acid methyl ester; and
(b) hydroxylamination of suberanilic acid methyl ester, to obtain SAHA.

16. The method of embodiment 15, further comprising a step of:
(c) non-chromatographic purification of SAHA.

17. The method of embodiment 15, wherein methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

18. The method of embodiment 15, wherein step (a) is carried out by reacting methyl-8-halogeno-8-oxooctanoate with aniline in the presence of a first alkaline catalyst at a molar ratio of methyl-8-halogeno-8-oxooctanoate: aniline: first alkaline catalyst of about 1:(1-2):(1-2).

19. The method of embodiment 18, wherein the first alkaline catalyst is triethylamine.

20 The method of embodiment 15, wherein step (b) is carried out by reacting suberanilic acid methyl ester with hydroxylamine in the presence of a second alkaline catalyst at a molar ratio of theoretical suberanilic acid methyl ester: hydroxylamine: second alkaline catalyst of about 1:(1-10):(1-10).

21. The method of embodiment 20, wherein the second alkaline catalyst is sodium methylate.

22. The method of embodiment 15, wherein step (a) comprises no chromatographic purification or non-chromatographic purification of intermediate obtained in the step.

23. A method for synthesizing high-purity SAHA, the method comprising:
(a) mixing suberic acid monomethyl ester with a halogenation reagent to obtain methyl-8-halogeno-8-oxooctanoate;
(b) adding a mixture of aniline and a first alkaline catalyst into (a) to obtain suberanilic acid methyl ester;
(c) adding suberanilic acid methyl ester into a mixture of a second alkaline catalyst and hydroxylamine to obtain SAHA having a purity of at least 90%; and
(d) optionally, purifying the obtained SAHA of step (c) via non-chromatographic purification to obtain high-purity SAHA having a purity of at least 99%.

24. The method of embodiment 23, wherein in step (a), suberic acid monomethyl ester and the halogenation reagent are mixed at a molar ratio of suberic acid monomethyl ester: halogenation reagent of about 1:(1-2).

25. The method of embodiment 23, wherein the halogenation reagent is selected from chlorine (Cl₂), thionyl chloride (SOCl₂), methanesulfonyl chloride, trichloromethanesulfonyl chloride, tert-butyl hypochlorite, dichloromethyl methyl ether, methoxyacetyl chloride, oxalyl chloride ((COCl)₂), phosphorus pentachloride (PCl₅), phosphorus trichloride (PCl₃), and trimethylsilyl chloride.

26 The method of embodiment 23, wherein the halogenation reagent is SOCl₂.

27. The method of embodiment 23, wherein in step (b), the mixture is added into methyl-8-halogeno-8-oxooctanoate to obtain a molar ratio of methyl-8-halogeno-8-oxooctanoate: aniline: first alkaline catalyst of about 1:(1-2):(1-2).

28. The method of embodiment 23, wherein the first alkaline catalyst is triethylamine.

29. The method of embodiment 23, wherein methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

30 The method of embodiment 23, wherein in step (c), suberanilic acid methyl ester is added into the mixture to reach a molar ratio of suberanilic acid methyl ester: hydroxylamine: second alkaline catalyst of about 1:(1-10):(1-10).

31. The method of embodiment 23, wherein the second alkaline catalyst is sodium methylate.

32. Suberoylanilide hydroxamic acid (SAHA) produced by the method of any one of embodiments 1-31.

33. A pharmaceutical composition comprising SAHA according to embodiment 32 and a pharmaceutically acceptable excipient.

34. The pharmaceutical composition of embodiment 33, wherein the pharmaceutically acceptable excipient is microcrystalline cellulose, sodium croscarmellose, magnesium stearate or combinations thereof.

35. The pharmaceutical composition of embodiment 33, wherein the pharmaceutically acceptable excipient is aqueous or non-aqueous.

36. The pharmaceutical composition of embodiment 33, wherein the pharmaceutically acceptable excipient is a syrup.

37. The pharmaceutical composition of embodiment 33, wherein the pharmaceutical composition comprises a diluent, antioxidant, preservative, coloring agent, flavoring agent, diluting agent, emulsifying agent, suspending agent, solvent, filler, bulking agent, buffer, delivery vehicle, tonicity agent, cosolvent, wetting agent, complexing agents buffering agent, antimicrobial, and/or surfactant.

### EXAMPLES

### Materials and Equipment

### Materials: Suberic Acid Monomethyl Ester (A)

Thionyl Chloride
Toluene
Aniline
Triethylamine
Tetrahydrofuran (THF)
Hydroxylamine Hydrochloride
Methanol
Sodium Methylate
Argon or Nitrogen (gas)

### Equipment: HPLC: Agilent 1200

QTOF-MS: Agilent 6545 QTOF-MS, Dual ESI
FTIR: Shimadzu IR Prestige-21
Coulometric Karl Fischer Tirando
NMR: Bruker Fourier 80 NMR

### Synthesis of Vorinostat

The synthetic preparation of Vorinostat is evaluated and optimized. The two-step synthesis of Vorinostat starts with the conversion of suberic acid monomethyl ester (compound A) to an acid chloride with thionyl chloride in toluene. The Methyl 8-Chloro-8-Oxooctanoate product (compound B) is obtained after rotary evaporation. Compound B is dissolved in tetrahydrofuran (THF) and mixed with aniline and triethylamine in a refrigerated and inert atmosphere to produce Suberanilic Acid Methyl Ester (compound C). In the second step, compound C dissolved in methanol is added to hydroxylamine hydrochloride and sodium methylate in a refrigerated and inert atmosphere. The final product is Suberoylanilide Hydroxamic Acid (SAHA), Vorinostat. The final product is dried under vacuum, re-crystallized, filtered, and washed to obtain a white to off-white, crystalline solid at room temperature. All steps in this synthetic sequence produced good yields.

### Example 1—Synthesis of Vorinostat

### Step 1: Preparation of Suberanilic Acid Methyl Ester (C)

To a mixture of suberic acid monomethyl ester (A) (60.25 g, 0.32 mol) and 60.0 mL dry toluene in a 1.0 L round bottom flask (RBF) equipped with condenser, thionyl chloride (30.0 mL, 0.41 mol) was added. The resulting mix was then stirred and heated on an oil bath below 60° C. overnight under an inert atmosphere of Argon. After ˜15 hours, the volatiles were removed on a rotary evaporator and residual oil, methyl-8-chloro-oxooctanoate (B), thus obtained, was dissolved in ˜600 mL of dry THF. The resulting solution was stirred and cooled in an ice-water bath below 5° C. under an argon blanket, and then a mixture of aniline (30.73 g, 0.33 mol) and triethylamine (46.0 mL, 0.33 mol) was added to it drop-wise, while keeping the temperature below 10° C. Once the addition was complete, the reaction mixture was stirred overnight at room temperature. The solid by-product, triethylamine hydrochloride, was filtered and then washed with about 150 mL of THF. The THF solution of the crude product, suberanilic acid methyl ester (C), was concentrated on a rotary evaporator at ˜60° C. and the residue was immediately dissolved in ˜75 mL methanol.

### Step 2: Preparation of Suberoylanilide Hydroxamic Acid (SAHA)

Hydroxylamine hydrochloride (111.20 g, 1.6 mol) and 600 mL methanol were taken in a 3-neck, 2.0 L RBF equipped with a magnetic stir bar and thermometer. The resulting mixture was stirred and cooled in an ice-water bath below 5° C. under an argon blanket, and then sodium methylate solution 25% in methanol (400 mL, 1.74 mol) was added drop-wise, keeping the temperature below 10° C. Once the addition was complete, the ice-water bath was removed. When the temperature of the reaction mixture reached about 15° C., the solution of crude suberanilic acid methyl ester was added. The resulting mixture was stirred overnight (˜15 hours) at room temperature. Using a cannula, the contents of the flask were added to a flask containing 2.5 L of ice-cold water being mechanically stirred in a 5.0 L, 3-neck RBF. The solid product was collected on a frit and washed with copious amounts of water, followed by a wash with ethyl acetate. The crude product was dried under high vacuum to give 76.5 g (90.4% yield). 73.0 g of crude product was further purified by crystallization from a mixture of 500 mL each of methanol and ethyl acetate. The product was filtered and washed with 8:2 (ethyl acetate: methanol) followed by ethyl acetate. The product was dried to give 65.5 g (90.0% yield).

### Example 2—Mass Spectral Analysis for Structural Characterization

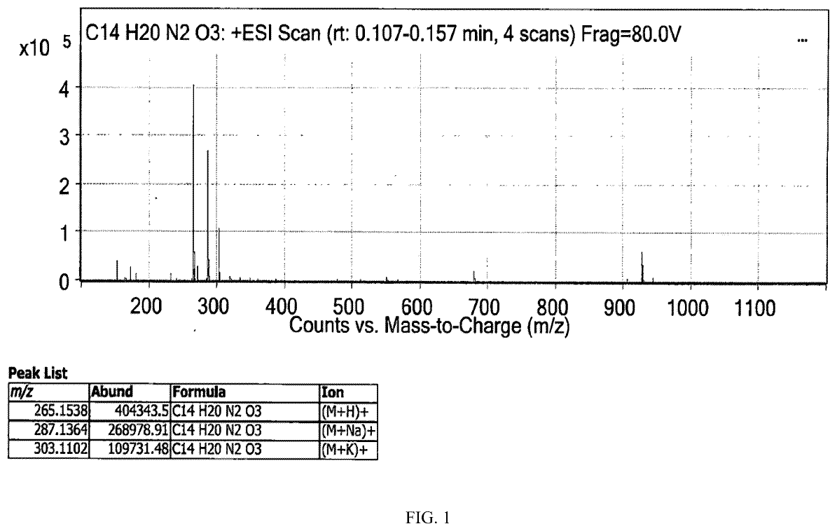

High-resolution mass spectrometry was performed by direct injection of the sample into an Agilent 6545 QTOF-MS, Dual ESI using positive electrospray ionization. The mass spectrum results are shown in Table 1. The mass spectrum of the Vorinostat synthesized by the present invention is consistent with the molecular formula of C14H20N2O3 for Vorinostat. The QTOF-MS spectrum is shown in FIG. 1.

TABLE 1
Mass Spectrum Results for Vorinostat Lot QCL-YS-041724

| | Major | | | Theoretical | Measured | Absolute Mass |
|---|---|---|---|---|---|---|
| Lot No./ | Monoisotopic | | Molecular | Monoisotopic | Monoisotopic | Matching Error |
| Preparation | Ion (m/z) | Ion Form | Formula for M | Mass for M | Mass for M | (ppm) |
| QCL-YS- | 265.1538 | [M + H]⁺ | C₁₄H₂₀N₂O₃ | 264.1474 | 264.1465 | <3.5 |
| 041724 | 287.1364 | [M + Na]⁺ | | | 264.1471 | <1.5 |
| Preparation 1 | 303.1102 | [M + K]⁺ | | | 264.1471 | <1.5 |
| QCL-YS- | 265.1544 | [M + H]⁺ | C₁₄H₂₀N₂O₃ | 264.1474 | 264.1472 | <1.0 |
| 041724 | 287.1355 | [M + Na]⁺ | | | 264.1463 | <4.5 |
| Preparation 2 | 303.1106 | [M + K]⁺ | | | 264.1475 | <0.5 |

### Example 3—Proton (¹H) NMR Spectroscopy for Structural Characterization

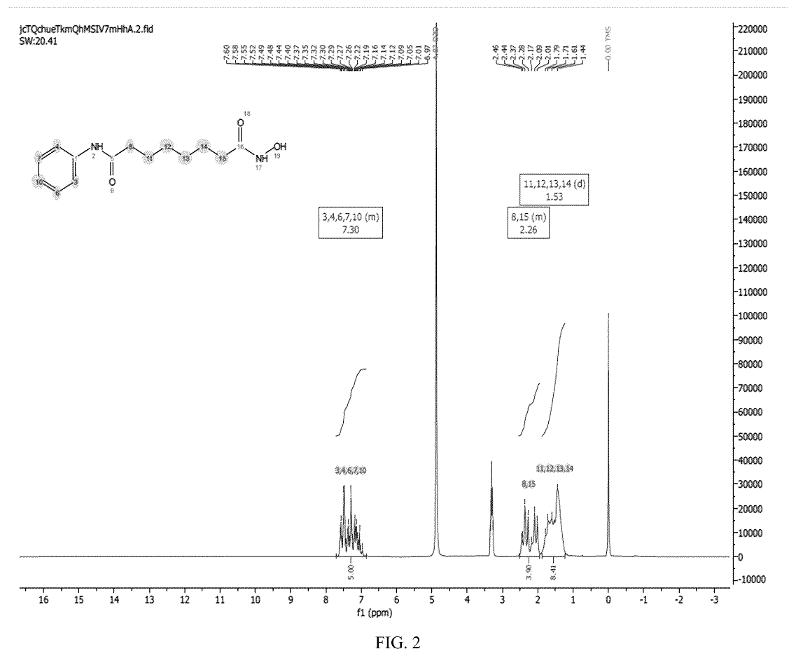

The ¹H-NMR spectra obtained with a Bruker Fourier 80 NMR spectrometer with the material in Methanol-d4/TMS are shown in FIG. 2. The results for the synthesized lot of material exhibits chemical shifts and multiplicity that are consistent with the structure for Vorinostat.

### Example 4—FTIR Spectroscopy for Structural Characterization

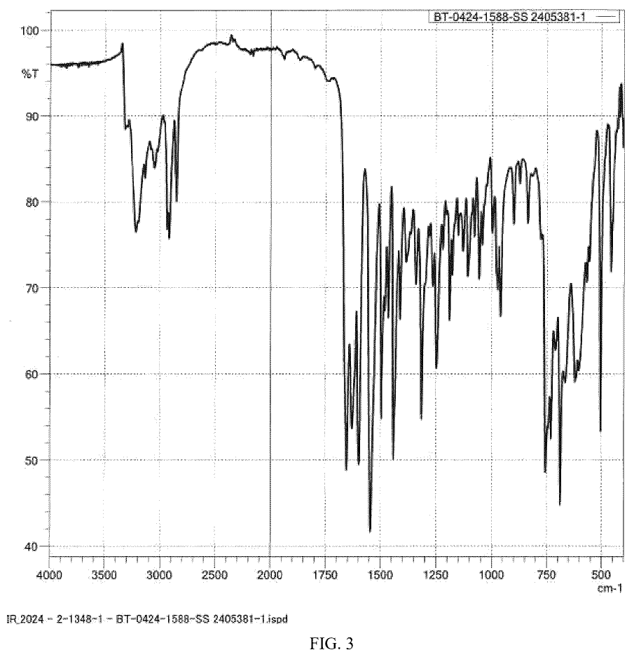

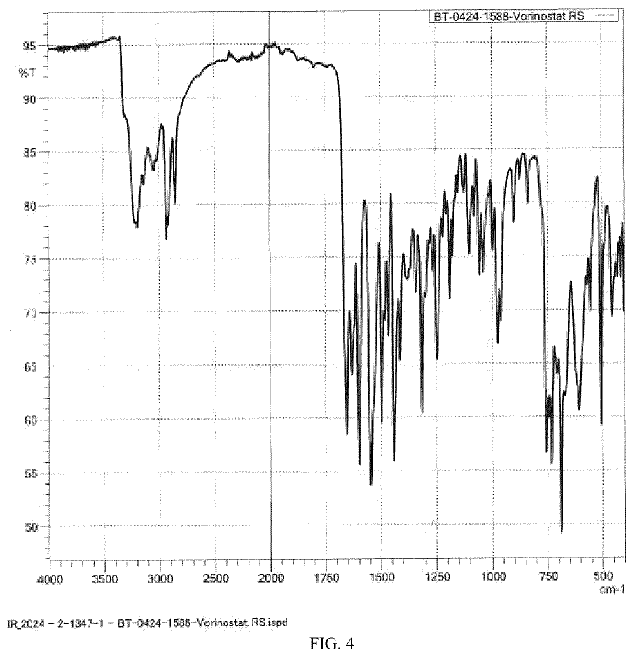

An FTIR spectrum of each lot of material was acquired using attenuated total reflection (ATR). The FTIR spectrum is shown in FIG. 3. The spectrum of the synthesized Vorinostat is consistent with the structure and functional groups in Vorinostat. The sample spectrum, when compared to the spectrum generated with Ambeed reference material, Lot A234507-AA8 shown in FIG. 4, had corresponding minima/maxima. The spectral assignments are shown in Table 2.

TABLE 2
FTIR Peak Spectral Assignments for
Vorinostat Lot QCL-YS-041724

| Wavelength (cm⁻¹) | Spectral Assignment |
|---|---|
| 3300-3200 | OH Stretching |
| | NH Stretching |
| 3000-2850 | C—H Stretching |
| 1654 | C═O Stretching |
| 1550 | |
| 1629 | C—H Aromatic |

### Example 5—Physical Characteristic

Description determination of the appearance was performed by viewing the sample against white and black backgrounds. The appearance results are shown in Table 3.

TABLE 3
Appearance Results for Vorinostat Lot QCL-YS-041724

| Lot No. | Result |
|---|---|
| QCL-YS-041724 | White, crystalline Powder |

### Example 6—Chromatographic Purity

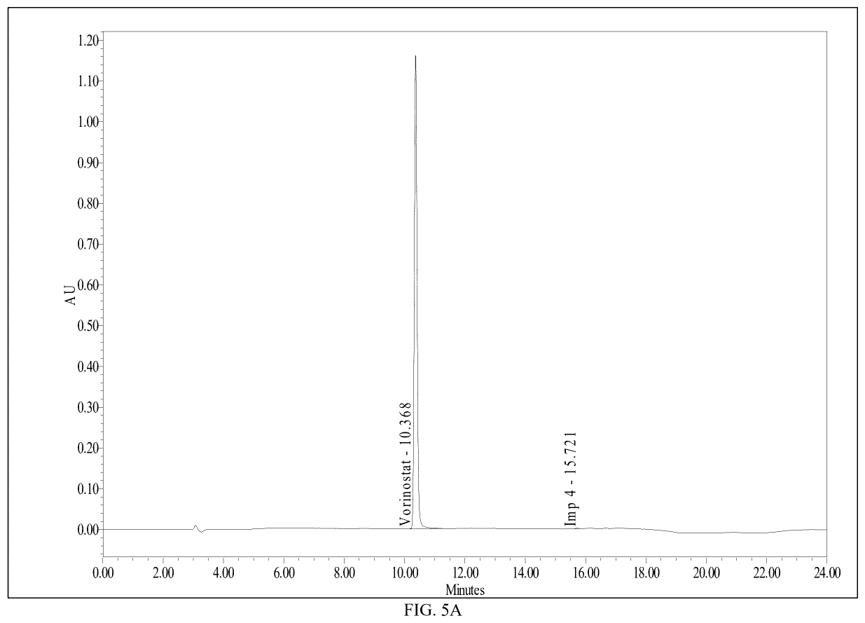

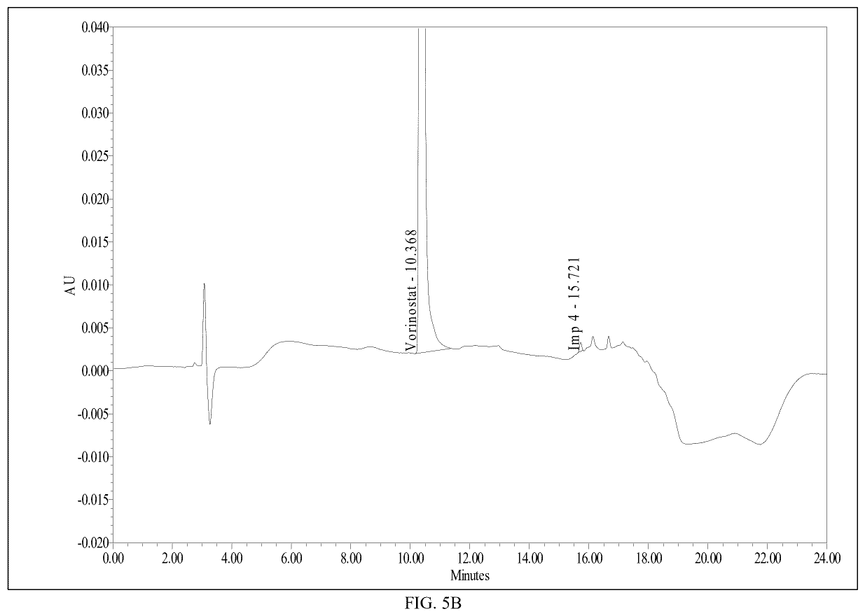

Chromatographic purity was determined by an HPLC analysis. The method is a reverse-phase HPLC gradient with UV detection at 242 nm. Solutions of the samples were prepared at 0.25 mg/mL in 1:1 (water: acetonitrile). 10 μL of the sample solution was injected on the HPLC system and separated using a Waters Xterra MS C18 250 mm×4.6 mm, 5 μm. The purity value was calculated using peak area percent. The chromatographic purity results and all impurities ≥0.05% are reported in Table 4. The off-scale and on-scale chromatograms are shown in FIGS. 5A-5B.

TABLE 4
Chromatographic Purity Results for
Vorinostat Lot QCL-YS-041724

| Lot No. | Purity (%) | Impurities |
|---|---|---|
| QCL-YS-041724 | 99.93 | RRT 1.52/0.07% |
| | | Total: 0.07% |

### Example 7—Moisture by Coulometric Karl Fischer

The moisture content of Vorinostat was determined by a coulometric Karl Fischer (cKF). The sample was accurately weighed (15 mg) into a preconditioned cKF vessel and titrated to an endpoint with coulomat AG. The moisture determination results are shown in Table 5.

TABLE 5
Moisture Results for Vorinostat Lot QCL-YS-041724

| Preparation | % Water |
|---|---|
| 1 | 0.50 |
| 3 | 0.19 |
| 2 | 0.13 |
| Mean (2) | 0.27 |

### Example 8—Characterization Summary

The synthesized Vorinostat sample was characterized using a variety of analytical techniques including high resolution quadrupole time-of-flight mass spectrometry (QTOF-MS), proton nuclear magnetic resonance spectroscopy (¹H-NMR), and Fourier transform infra-red spectroscopy (FTIR). Additionally, the appearance of the material was described, the purity determined by HPLC, and the moisture was determined by coulometric Karl Fischer.

TABLE 6
Summary Table Vorinostat Lot

| Test Description | Result |
|---|---|
| Appearance | White crystalline powder |
| Coulometric Karl Fischer | Preparation 1: 0.50% |
| | Preparation 2: 0.19% |
| | Preparation 3: 0.13% |
| | Mean (3): 0.27% |
| Identification by IR | The IR spectrum is consistent with |
| | the reference material spectrum and |
| | chemical structure of Vorinostat |
| Identification by ¹H NMR | ¹H NMR is consistent with the |
| | chemical structure of Vorinostat |
| Mass Spectroscopy | The mass spectrum is consistent with |
| | the chemical structure of Vorinostat |
| | (C₁₄H₂₀N₂O₃) |
| Chromatographic Purity | Purity: 99.93% |
| | Unknown Impurity: RRT 1.52/0.07% |
| | Total Impurities: 0.07% |

Vorinostat was successfully synthesized in the present invention. The synthetic scheme yielded a high purity product that would be suitable for GMP manufacturing scale-up. All analytical data obtained is included in the present invention. The overall synthesis described herein has not been reported and does not require purifying any of the intermediates and produces the final product in high yields and high purity.

### Example 9—Ames Study of Compound RVL001 & ZOLINZA®

### 1. Study Purpose

The purpose of this study was to evaluate the mutagenicity potential of test articles compounds RVL001 & ZOLINZA® the AMES reverse mutation assay in the presence or absence of rat liver S9 fraction. This study was performed under non-GLP conditions.

RVL001 represents the Vorinostat lot that was synthesized according to the method described herein, and ZOLINZA® is a commercially available Vorinostat product from Merck manufactured using their proprietary synthesis process, see, for example, U.S. Pat. Nos. 7,456,219, 8,093,295, 7,652,069, 7,851,509, and 8,450,372 (each of which is hereby incorporated by reference in its entirety.

### 2. Experimental Design

### A. Solubility Test-COMP RVL001 & ZOLINZA®

RVL001 & ZOLINZA® stock in DMSO at 50 mg/mL (soluble) was first made. Then the solubility of RVL001 & ZOLINZA® was tested from 2 mg/mL to 62.5 μg/mL in HBSS buffer.

Precipitates are visible for RVL001 under the inverted microscope at 2 mg/mL. No precipitates are visible for RVL001 at 1 mg/mL, 500 μg/mL, 250 μg/mL, 125 μg/mL and 62.5 μg/mL.

The precipitates are visible for ZOLINZA® at 2 mg/mL, small amount of precipitate is visible at 1 mg/mL and no precipitates are visible at 500 μg/mL, 250 μg/mL, 125 μg/mL, 62.5 μg/mL.

Six concentrations were tested at 2 mg/mL, 1 mg/mL, 500 μg/mL, 250 μg/mL, 125 μg/mL and 62.5 mg/ml for both compounds.

### B. Experimental Conditions

TABLE 7
Experimental Conditions

| | Test Concentrations* | |
|---|---|---|
| Test Article | (μg/mL) | Reference Compounds |
| RVL001 | 62.5, 125, 250, 500, 1000, | 2-nitrofluorene |
| | 2000 (μg/mL) | 4-nitroquinoline N-oxide |
| | | 2-aminoanthracene |
| ZOLINZA ® | 62.5, 125, 250, 500, 1000, | 2-nitrofluorene |
| | 2000 (μg/mL) | 4-nitroquinoline N-oxide |
| | | 2-aminoanthracene |

### C. Experiment Procedure

Bacteria were exposed in triplicate to six concentrations of the test compound COMP RVL001 & ZOLINZA®, a negative control (vehicle) and a positive control for 90 min in medium containing a low concentration of histidine to support approximately two cell divisions. The cultures were then diluted into pH indicator medium lacking histidine, and dispensed into 48 wells of a 384 well plate. The plates were incubated for 48 hrs at 37° C. and cells that underwent a reversion growth in the well, resulting in a color change during growth (from blue to yellow). The number of wells containing revertant colonies were counted and compared to the vehicle control. When S9 fraction was included in the experiment, S9 fraction from Rat liver S9 mix were included in the incubation at a final concentration of 4.5%. An NADPH regenerating system was included as well to ensure a steady supply of reducing equivalents.

An increase in the number of revertant colonies of at least two-fold over baseline (mean±SD of the vehicle control) and a dose response indicate a positive response. An unpaired, one-sided Student's T-test was used to identify the conditions that were significantly different from the vehicle control.

Strains used in this study:
S. typhimurium TA98: hisD3052, rfa, uvrB/pKM101; detects frame-shift mutations.
S. typhimurium TA100: hisG46, rfa, uvrB/pKM101; detects base-pair substitutions.

### 3. Results

RVL001 & ZOLINZA® were assessed for their mutagenic potential in the AMES reverse mutation assay at concentration 0, 62.5 μg/mL, 125 μg/mL, 250 μg/mL, 500 μg/mL, 1000 μg/mL and 2000 μg/mL. This test was performed in the absence and presence of Rat S9 metabolic activation.

RVL001 does not exhibit any genotoxic effects against either strain in this assay. Although RVL001 2000 μg/ml shows a 2.05 fold increase against TA100 in the absence of S9 but the p value shows no significance.

ZOLINZA® shows positive genotoxic effects against TA100 in the absence of S9 (at 125 μg/ml, 250 μg/ml, 500 μg/ml and 1000 μg/ml concentrations) and in the presence of S9 (at 1000 μg/mL and 2000 μg/mL concentrations).

### A. Data Summary

TABLE 8
Data Summary

| | | | | Highest | |
|---|---|---|---|---|---|
| | Test | | AMES Result | Conc. | |
| Test Article | Strain | S9 | (Positive/Negative) | Tested* | Comment |
| 2-nitrofluorine | TA98 | No | Positive | 4 | positive control |
| | | | | μg/mL | |
| 4-nitroquinoline | TA100 | No | Positive | 0.1 | positive control |
| N-oxide | | | | μg/mL | |
| 2-aminoanthracene | TA98 | Yes | Positive | 2.5 | positive control |
| | | | | μg/mL | |
| 2-aminoanthracene | TA100 | Yes | Positive | 2.5 | positive control |
| | | | | μg/mL | |
| RVL001 | TA98 | No | Negative | 2000 | No genotoxicity |
| | | | | μg/mL | observed |
| | TA100 | No | Negative | 2000 | |
| | | | | μg/mL | |
| | TA98 | Yes | Negative | 2000 | |
| | | | | μg/mL | |
| | TA100 | Yes | Negative | 2000 | |
| | | | | μg/mL | |
| ZOLINZA ® | TA98 | No | Negative | 2000 | No genotoxicity |
| | | | | μg/mL | observed |
| | TA100 | No | Positive response | 2000 | Genotoxicity |
| | | | observed | μg/mL | observed |
| | | | at 125 ug/ml, 250 ug/ml. | | |
| | | | 500 ug/ml and | | |
| | | | 1000 ug/ml. | | |
| | TA98 | Yes | Negative | 2000 | No genotoxicity |
| | | | | μg/mL | observed |
| | TA100 | Yes | Positive response | 2000 | Genotoxicity |
| | | | observed at 1000 ug/ml | μg/mL | observed |
| | | | and | | |
| | | | 2000 ug/ml. | | |

*Highest concentration of the test article

### B. Individual Data

Significant fold increase over baseline values (≥2-fold) are indicated with *. RVL001 (Tables 9 to 12); ZOLINZA® (Tables 13 to 16).

TABLE 9
Individual Data of RVL001

| Compound: RVL001 | | | | | Fold | |
|---|---|---|---|---|---|---|
| TA98 − S9 | | mean # | | | increase | |
| Concentration | | pos. | | | (over | t-test |
| (μg/ml) | n | Wells | SD | Baseline | baseline) | p-value |
| 0 | 3 | 0.67 | 0.58 | 1.24 | | |
| 62.50 | 3 | 0.33 | 0.58 | | 0.27 | 0.26 |
| 125 | 3 | 0.67 | 0.58 | | 0.54 | 0.26 |
| 250 | 3 | 0.00 | 0.00 | | 0.00 | 0.09 |
| 500 | 3 | 0.67 | 1.15 | | 0.54 | 0.21 |
| 1000 | 3 | 0.67 | 1.15 | | 0.54 | 0.50 |
| 2000 | 3 | 0.00 | 0.00 | | 0.00 | 0.21 |
| Pos. contr: 2- | 3 | 43.33 | 0.58 | | 34.83* | 0.00 |
| NF | | | | | | |

TABLE 10
Individual Data of RVL001

| Compound: RVL001 | | | | | Fold | |
|---|---|---|---|---|---|---|
| TA98 + S9 | | mean # | | | increase | |
| Concentration | | pos. | | | (over | t-test |
| (μg/ml) | n | Wells | SD | Baseline | baseline) | p-value |
| 0 | 3 | 2.00 | 1.73 | 3.73 | | |
| 62.50 | 3 | 2.00 | 2.00 | | 0.54 | 0.50 |
| 125 | 3 | 4.00 | 1.00 | | 1.07 | 0.09 |
| 250 | 3 | 4.67 | 1.53 | | 1.07 | 0.28 |
| 500 | 3 | 4.00 | 1.73 | | 1.07 | 0.32 |
| 1000 | 3 | 2.67 | 1.53 | | 0.71 | 0.19 |
| 2000 | 3 | 3.33 | 2.89 | | 0.89 | 0.37 |
| Post. contr: 2-AA | 3 | 44.67 | 0.58 | | 11.97* | 0.00* |

TABLE 11
Individual Data of RVL001

| Compound: RVL001 | | | | | Fold | |
|---|---|---|---|---|---|---|
| TA100 − S9 | | mean # | | | increase | |
| Concentration | | pos. | | | (over | t-test |
| (μg/ml) | n | Wells | SD | Baseline | baseline) | p-value |
| 0 | 3 | 3.67 | 1.53 | 5.19 | | |
| 62.50 | 3 | 5.33 | 0.58 | | 1.03 | 0.10 |
| 125 | 3 | 4.33 | 1.15 | | 0.83 | 0.29 |
| 250 | 3 | 3.67 | 1.15 | | 0.71 | 0.50 |
| 500 | 3 | 4.00 | 1.00 | | 0.77 | 0.39 |
| 1000 | 3 | 6.00 | 1.00 | | 1.16 | 0.05 |
| 2000 | 3 | 10.67 | 4.93 | | 2.05* | 0.10 |
| Pos. contr: 4- | 3 | 48.00 | 0.00 | | 9.24* | 0.00* |
| NQO | | | | | | |

TABLE 12
Individual Data of RVL001

| Compound: RVL001 | | | | | Fold | |
|---|---|---|---|---|---|---|
| TA100 + S9 | | mean # | | | increase | |
| Concentration | | pos. | | | (over | t-test |
| (μg/ml) | n | Wells | SD | Baseline | baseline) | p-value |
| 0 | 3 | 4.67 | 0.58 | 5.24 | | |
| 62.50 | 3 | 5.00 | 1.00 | | 0.95 | 0.32 |
| 125 | 3 | 4.67 | 1.53 | | 0.89 | 0.50 |
| 250 | 3 | 5.67 | 1.15 | | 1.08 | 0.14 |
| 500 | 3 | 5.33 | 1.53 | | 1.02 | 0.27 |
| 1000 | 3 | 7.00 | 1.00 | | 1.33 | 0.02* |
| 2000 | 3 | 4.67 | 2.31 | | 0.89 | 0.50 |
| Pos. contr: 2-AA | 3 | 45.67 | 1.53 | | 8.71* | 0.00* |

TABLE 13
Individual Data of ZOLINZA ®

| Compound: ZOLINZA ® | | | | | Fold | |
|---|---|---|---|---|---|---|
| TA98 − S9 | | mean # | | | increase | |
| Concentration | | pos. | | | (over | t-test |
| (μg/ml) | n | Wells | SD | Baseline | baseline) | p-value |
| 0 | 3 | 1.00 | 1.00 | 2.00 | | |
| 62.50 | 3 | 1.00 | 1.00 | | 0.50 | 0.5000 |
| 125 | 3 | 0.33 | 0.58 | | 0.17 | 0.1934 |
| 250 | 3 | 0.00 | 0.00 | | 0.00 | 0.2113 |
| 500 | 3 | 0.67 | 0.58 | | 0.33 | 0.0918 |
| 1000 | 3 | 2.33 | 1.53 | | 1.17 | 0.1423 |
| 2000 | 3 | 0.00 | 0.00 | | 0.00 | 0.0590 |
| Pos. contr: 2-NF | 3 | 43.33 | 1.53 | | 21.67* | 0.0002 |

TABLE 14
Individual Data of ZOLINZA ®

| Compound: ZOLINZA ® | | | | | Fold | |
|---|---|---|---|---|---|---|
| TA98 + S9 | | mean # | | | increase | |
| Concentration | | pos. | | | (over | t-test |
| (μg/ml) | n | Wells | SD | Baseline | baseline) | p-value |
| 0 | 3 | 2.67 | 1.15 | 3.82 | | |
| 62.50 | 3 | 3.33 | 0.58 | | 0.87 | 0.219097 |
| 125 | 3 | 3.00 | 2.00 | | 0.79 | 0.4089 |
| 250 | 3 | 3.00 | 1.00 | | 0.79 | 0.5000 |
| 500 | 3 | 5.33 | 1.15 | | 1.40 | 0.0292 |
| 1000 | 3 | 6.00 | 1.00 | | 1.57 | 0.2463 |
| 2000 | 3 | 10.67 | 4.73 | | 2.79* | 0.1130 |
| Post. contr: | 3 | 43.67 | 1.53 | | 11.43* | 0.0000* |
| 2-AA | | | | | | |

TABLE 15
Individual Data of ZOLINZA ®

| Compound: ZOLINZA | | | | | | |
|---|---|---|---|---|---|---|
| TA100 − S9 | | | | | Fold | |
| | | mean # | | | increase | |
| Concentration | | pos. | | | (over | t-test |
| (μg/ml) | n | Wells | SD | Baseline | baseline) | p-value |
| 0 | 3 | 4.00 | 1.73 | 5.73 | | |
| 62.50 | 3 | 6.67 | 1.53 | | 1.16 | 0.05861 |
| 125 | 3 | 11.67 | 0.58 | | 2.04* | 0.0052* |
| 250 | 3 | 13.67 | 0.58 | | 2.38* | 0.0030* |
| 500 | 3 | 15.33 | 0.58 | | 2.68* | 0.0020* |
| 1000 | 3 | 15.67 | 0.58 | | 2.73* | 0.0019* |
| 2000 | 3 | 4.33 | 1.53 | | 0.76 | 0.0674 |
| Pos. contr: 4- | 3 | 48.00 | 0.00 | | 8.37* | 0.0003* |
| NQO | | | | | | |

TABLE 16
Individual Data of ZOLINZA ®

| Compound: ZOLINZA ® | | | | | Fold | |
|---|---|---|---|---|---|---|
| TA100 + S9 | | mean # | | | increase | |
| Concentration | | pos. | | | (over | t-test |
| (μg/ml) | n | Wells | SD | Baseline | baseline) | p-value |
| 0 | 3 | 4.33 | 0.58 | 4.91 | | |
| 62.50 | 3 | 5.33 | 2.08 | | 1.09 | 0.2485 |
| 125 | 3 | 6.33 | 1.53 | | 1.29 | 0.0697 |
| 250 | 3 | 6.00 | 1.00 | | 1.22 | 0.0412 |
| 500 | 3 | 3.67 | 0.58 | | 0.75 | 0.1151 |
| 1000 | 3 | 15.67 | 0.58 | | 3.19* | 0.0000* |
| 2000 | 3 | 16.00 | 0.00 | | 3.26* | 0.0004* |
| Pos. contr: 2-AA | 3 | 47.33 | 1.15 | | 9.64* | 0.0000* |

### 4. Discussion

Results have shown that the very pure synthesis process of Vorinostat provided in the present invention (the compound designated RVL001) results in a negative Ames test for mutagenicity. The additional fact that the mutagenicity is observed in ZOLINZA®, Merck's Vorinostat, but not the Vorinostat product synthesized in the present invention (RVL001) suggests that the observed toxicity may result primarily from contaminants/byproducts and not Vorinostat directly. Especially since with and without the S9 liver fraction addition the same results were obtained (i.e., mutagenesis was not caused by a metabolite). This finding is notable in that it suggests that the input drug substance and not downstream metabolites are at the root of potential genotoxicity. While it has been known that the hydroxamic acid moiety may be genotoxic, the results here suggest that synthesis byproducts, which may or may not contain hydroxamic acid groups, are significant contributors to genotoxicity. This indicates that the synthesis process described in this invention enables a higher purity compound to be produced that results in an unexpectedly lower genotoxicity when measured using an Ames test. The described invention offers potentially lower risk to patients despite identical active molecular structures of the active compounds. From a regulatory perspective, this also means that vorinostat, if synthesized to a sufficient purity, may pose a lower risk to patients than previously assumed based on low purity vorinostat drug substance data. This synthesis method's high purity may accelerate regulatory review and approval and time to market.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

What is claimed is:

1. A method for preparing suberoylanilide hydroxamic acid (SAHA) from suberic acid monomethyl ester, comprising steps of:
(a) halogenation of suberic acid monomethyl ester, to obtain methyl-8-halogeno-8-oxooctanoate;
(b) amidation of methyl-8-halogeno-8-oxooctanoate, to obtain suberanilic acid methyl ester; and
(c) hydroxylamination of suberanilic acid methyl ester, to obtain SAHA.

2. The method of claim 1, further comprising a step of:
(d) non-chromatographic purification of SAHA.

3. The method of claim 1, wherein step (a) is carried out by reacting suberic acid monomethyl ester with a halogenation reagent at a molar ratio of suberic acid monomethyl ester: halogenation reagent of about 1:(1-2).

4. The method of claim 3, wherein the halogenation reagent is selected from chlorine (Cl₂), thionyl chloride (SOCl₂), methanesulfonyl chloride, trichloromethanesulfonyl chloride, tert-butyl hypochlorite, dichloromethyl methyl ether, methoxyacetyl chloride, oxalyl chloride ((COCl)₂), phosphorus pentachloride (PCl₅), phosphorus trichloride (PCl₃), and trimethylsilyl chloride.

5. The method of claim 4, wherein the halogenation reagent is SOCl₂.

6. The method of claim 1, wherein step (a) is carried out at a temperature of about 20-60° C.

7. The method of claim 1, wherein step (b) is carried out by reacting methyl-8-halogeno-8-oxooctanoate with aniline in the presence of a first alkaline catalyst at a molar ratio of methyl-8-halogeno-8-oxooctanoate: aniline: first alkaline catalyst of about 1:(1-2):(1-2).

8. The method of claim 7, wherein step (b) is carried out by firstly mixing the first alkaline catalyst with aniline, followed by adding the mixture of the first alkaline catalyst and aniline into methyl-8-halogeno-8-oxooctanoate.

9. The method of claim 7, wherein the first alkaline catalyst is triethylamine.

10. The method of claim 1, wherein step (c) is carried out by reacting suberanilic acid methyl ester with hydroxylamine in the presence of a second alkaline catalyst at a molar ratio of suberanilic acid methyl ester: hydroxylamine: second alkaline catalyst of about 1:(1-10):(1-10).

11. The method of claim 10, wherein step (c) is carried out by firstly mixing the second alkaline catalyst with hydroxylamine, followed by adding suberanilic acid methyl ester into the mixture of the second alkaline catalyst and hydroxylamine.

12. The method of claim 10, wherein the second alkaline catalyst is sodium methylate.

13. The method of claim 1, wherein step (a) and step (b) comprise no chromatographic purification or non-chromatographic purification of intermediate obtained in each step.

14. The method of claim 1, wherein methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

15. A method for preparing SAHA from methyl-8-halogeno-8-oxooctanoate, comprising steps of:
(a) amidation of methyl-8-halogeno-8-oxooctanoate, to obtain suberanilic acid methyl ester; and
(b) hydroxylamination of suberanilic acid methyl ester, to obtain SAHA.

16. The method of claim 15, further comprising a step of:
(c) non-chromatographic purification of SAHA.

17. The method of claim 15, wherein methyl-8-halogeno-8-oxooctanoate is selected from methyl-8-fluoro-8-oxooctanoate, methyl-8-chloro-8-oxooctanoate, methyl-8-bromo-8-oxooctanoate, and methyl-8-iodo-8-oxooctanoate.

18. A method for synthesizing high-purity SAHA, the method comprising:
(a) mixing suberic acid monomethyl ester with a halogenation reagent to obtain methyl-8-halogeno-8-oxooctanoate;
(b) adding a mixture of aniline and a first alkaline catalyst into (a) to obtain suberanilic acid methyl ester;
(c) adding suberanilic acid methyl ester into a mixture of a second alkaline catalyst and hydroxylamine to obtain SAHA having a purity of at least 90%; and
(d) optionally, purifying the obtained SAHA of step (c) via non-chromatographic purification to obtain high-purity SAHA having a purity of at least 99%.

19. Suberoylanilide hydroxamic acid (SAHA) produced by the method of claim 1.

20. A pharmaceutical composition comprising SAHA according to claim 19 and a pharmaceutically acceptable excipient.
